(19) United States
(12) Patent Application Publication
(10) Pub. No.: US 2013/0295203 A1
(43) Pub. Date: Nov. 7, 2013

(54) METHODS AND COMPOSITIONS FOR THE TREATMENT OF NEUROPSYCHIATRIC DISORDERS

(71) Applicant: Jay L. LOMBARD, New City, NY (US)

(72) Inventor: Jay L. LOMBARD, New City, NY (US)

(21) Appl. No.: 13/937,168

(22) Filed: Jul. 8, 2013

Related U.S. Application Data
(63) Continuation-in-part of application No. 13/210,808, filed on Aug. 16, 2011.
Application No. 13/210,808 is a continuation-in-part of application No. 13/365,076, filed on Feb. 2, 2012.
Application No. 13/365,076 is a continuation-in-part of application No. 13/739,970, filed on Jan. 11, 2013.
Application No. 13/739,970 is a continuation of application No. 13/371,227, filed on Feb. 10, 2012, now Pat. No. 8,355,927.
Application No. 13/371,227 is a continuation-in-part of application No. 13/290,603, filed on Nov. 7, 2011.
(60) Provisional application No. 61/669,423, filed on Jul. 9, 2012.
(60) Provisional application No. 61/674,240, filed on Jul. 20, 2012.
(60) Provisional application No. 61/674,247, filed on Jul. 20, 2012.
(60) Provisional application No. 61/693,740, filed on Aug. 27, 2012.
(60) Provisional application No. 61/705,100, filed on Sep. 24, 2012.
(60) Provisional application No. 61/705,095, filed on Sep. 24, 2012.
(60) Provisional application No. 61/841,902, filed on Jul. 1, 2013.
(60) Provisional application No. 61/374,225, filed on Aug. 16, 2010.
(60) Provisional application No. 61/438,924, filed on Feb. 2, 2011.
(60) Provisional application No. 61/410,523, filed on Nov. 5, 2010.
(60) Provisional application No. 61/528,583, filed on Aug. 29, 2011.

(51) Int. Cl. A61K 31/685; A61K 31/232; A61K 31/201; A61K 31/198; A61K 31/197; A61K 33/06; A61K 31/375; A61K 31/26; A61K 31/519; A61K 31/185

(52) U.S. Cl. CPC A61K 31/685; A61K 31/519; A61K 31/232; A61K 31/201; A61K 31/185; A61K 31/197; A61K 33/06; A61K 31/375; A61K 31/26; A61K 31/198
USPC 424/682; 514/77; 514/515

(57) ABSTRACT

Methods and compositions are disclosed to treat neuropsychiatric disorders based upon a new framework of diagnosis. Axis I biomarkers include genes related to prefrontal dopamine synthesis and/or dopamine degradation. Axis II includes genes related to re uptake of dopamine, norepinephrine and serotonin and autonomic hyperactivity. Axis III includes genes relates to impairments in inflammatory pathways, glutamate neurotransmission and/or neurotrophic factors. Axis IV includes genes related to glutamate reuptake and predisposition to addictive behavior, and obsessive compulsions.

### CROSS REFERENCE TO RELATED APPLICATIONS

This patent application claims priority to the following provisional patent applications: U.S. Provisional Patent Application No. 61/669,423, filed Jul. 9, 2012, and titled “MEDICAL FOODS FOR THE TREATMENT OF SUBTYPES OF AUTISM AND METHODS OF USE;” U.S. Provisional Patent Application No. 61/674,240, filed Jul. 20, 2012, and titled “USE OF THE COMT ACTIVITY TO GUIDE TREATMENT OF SUBTYPES OF DEPRESSION AND CHRONIC PAIN SYNDROMES;” U.S. Provisional Patent Application No. 61/674,247, filed Jul. 20, 2012, and titled “COMPANION DIAGNOSTIC-BASED MEDICAL FOOD FOR TREATMENT OR PREVENTION OF DEMENTIA;” U.S. Provisional Patent Application No. 61/693,740, filed Aug. 27, 2012, and titled “COMPANION DIAGNOSTICS AND MEDICAL FOOD INTERVENTIONS IN NEUROPSYCHIATRY;” U.S. Provisional Patent Application No. 61/705,100, filed Sep. 24, 2012, and titled “COMPANION DIAGNOSTICS AND MEDICAL FOOD INTERVENTIONS IN NEUROPSYCHIATRY;” U.S. Provisional Patent Application No. 61/705,095, filed Sep. 24, 2012, and titled “SIALIC ACID COMPOUNDS FOR THE PREVENTION OR TREATMENT OF AUTISM AND PERVASIVE DEVELOPMENTAL DELAY;” and U.S. Provisional Patent Application No. 61/841,902, filed Jul. 1, 2013, and titled “METHODS AND COMPOSITIONS FOR THE TREATMENT OF NEUROPSYCHIATRIC DISORDERS,” each of which is herein incorporated by reference in its entirety.

This patent application is also a continuation-in-part of U.S. patent application Ser. No. 13/210,808, filed Aug. 16, 2011 and titled “MEDICAL FOODS FOR THE TREATMENT OF DEVELOPMENTALLY-BASED NEUROPSYCHIATRIC DISORDERS VIA MODULATION OF BRAIN GLYCINE AND GLUTATHIONE PATHWAYS,” Publication No. U.S.-2012-0041066-A1 which claims priority to U.S. provisional patent application Ser. No. 61/374,225, filed on Aug. 16, 2010, titled “MEDICAL FOODS FOR THE TREATMENT OF DEVELOPMENTALLY-BASED NEUROPSYCHIATRIC DISORDERS VIA MODULATION OF BRAIN GLYCINE AND GLUTATHIONE PATHWAYS,” each of which is herein incorporated by reference in its entirety.

This patent application is also a continuation-in-part of U.S. patent application Ser. No. 13/365,076, filed Feb. 2, 2012 and titled “DIAGNOSIS AND TREATMENT OF THE PRODROMAL SCHIZOPHRENIC STATE,” Publication No. U.S.-2012-0195984-A1, which claims priority to U.S. Provisional Patent Application No. 61/438,924, filed on Feb. 2, 2011, and titled “TREATMENT OF THE PRODROMAL SCHIZOPHRENIC STATE,” each of which is herein incorporated by reference in its entirety.

This patent application is also a continuation-in-part of U.S. patent application Ser. No. 13/739,970, filed Jan. 11, 2013 and titled “NEUROPSYCHIATRIC TEST REPORTS,” Publication No. U.S.-2013-0132114-A1 which is a continuation of U.S. patent application Ser. No. 13/371,227, Filed Feb. 10, 2012 and titled “NEUROPSYCHIATRIC TEST REPORTS,” now U.S. Pat. No. 8,355,927, which claims priority as a continuation-in-part to U.S. patent application Ser. No. 13/290,603, filed Nov. 7, 2011 and titled “NEUROPSYCHIATRIC TEST REPORTS,” Publication No. U.S.-2012-0115147-A1, which claims priority to U.S. Provisional Patent Application No. 61/410,523, filed Nov. 5, 2010, titled “TREATMENT RESISTANT DEPRESSION DIAGNOSTIC TEST REPORT,” each of which is herein incorporated by reference in its entirety.

U.S. patent application Ser. No. 13/371,227 also claims priority to U.S. Provisional Patent Application No. 61/528,583, filed Aug. 29, 2011 and titled “INTERPRETIVE BIOMARKER SCREENING REPORTS FOR DIAGNOSIS AND TREATMENT OF PSYCHIATRIC DISORDERS,” each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Personalized medicine for neuropsychiatric disorders.

### BACKGROUND

Most psychiatric classification systems, which are based upon categorical assessments, do not take into account that most mental disorders are dimensional with similar and overlapping symptoms in patients with discrete diagnostic categories. Virtually all brain disorders may cause psychiatric symptoms. The term “neuropsychiatric disorders” may refer to brain disease or dysfunction that causes psychiatric symptoms. Examples of neuropsychiatric disorders include depression (including treatment resistant depression, bipolar depression, etc . . . ), schizophrenia, PTSD and other anxiety disorders, autism, ADHD, and the like.

Personalized medicine is considered a young but rapidly advancing field of healthcare that is informed by each person's unique clinical, genomic, and environmental information. One goal of personalized medicine as used herein is to customize or individualize treatments by suggesting or providing a medical food based on the particular biomarkers specific to the patient. For example, the patient's genomic profile may allow accurate predictions as to what composition of a medical food a patient may respond to, or receive the most benefit from. There is an ever-growing need to understand what therapies, and in particular, what medical foods, would benefit a patient in a manner that is specific to that patient's needs. This disclosure provides a new way of assessing which medical foods are most appropriate for individuals based upon specific genetic patterns.

As used herein, a medical food is a composition that addresses a nutritional that you cannot otherwise be met from a patient's normal dietary sources. Thus a medical food may treat a specific need arising from a medical defect. As an example, a deficiency of an amino acid or fatty acid may be a consequence of a particular gene variant and be associated with psychiatric symptomatology. By addressing the underlying dietary deficiency resulting from such genetic variation, a specific medical food or foods may also alleviate the associated psychiatric symptoms. Medical foods may include food which is formulated to be consumed or administered enterally under the supervision of a physician and which is intended for the specific dietary management of a disease or condition for which distinctive nutritional requirements, based on recognized scientific principles, are established by medical evaluation. Medical foods are foods that are specially formulated and processed (as opposed to a naturally occurring foodstuff used in a natural state) for the patient who is ill or who may benefit from the product as a treatment modality. A medical food may be a food for oral or tube feeding. The medical food may be for the dietary management of a specific medical disorder, disease, or condition as described herein. The medical food may be used under medical supervision. In some variations, a medical food may be used as an over-the-counter composition.

In the last fifty years, a tremendous amount of research has begun to elucidate the causes, characteristics and treatments of many neuropsychiatric disorders. Unfortunately, this process has not efficiently translated into effective patient treatments. The term “treatment resistance” is unfortunately more the norm than the exception in psychiatry. For instance, in the Catie trials, which looked at the effectiveness of medications in schizophrenia, more than 75% of patients discontinued medication within 18 months, and in the Star-D trials more than 50% of patients with depression did not achieve remission despite the use of two or more antidepressants or use of an augmenting agent.

This “treatment resistance” may be due in part to the fact that neuropsychiatric disorders are notoriously difficult to diagnose because existing categories of disorders are imprecise; there is a great deal of overlap and comorbidity in these conditions. It is also relevant to note that in randomized clinical trials, a significant majority of patients (85%) would need to be excluded because of a comorbid psychiatric or medical condition. Thus, it is difficult to accurately identify patients properly for treatment, either clinically or in research settings. In general, categorical tests for neuropsychiatric disorders, such as DSM, have not proven effective in accurately diagnosing and treating patients, as there is a great deal of variation in patient outcomes between patients categorized with the same diagnosis. Most psychiatric classification systems, which are based upon categorical assessments, do not take into account that most mental disorders are dimensional with similar and overlapping symptoms in patients, rather than discrete diagnostic categories. Even in research settings, the limitations of current categorical nosology of psychiatric disorders results in a disconnect between trial findings and application to real world settings. This problem was recently addressed by the NIH, which has proposed an alternative classification system referred to as “research domain criteria”. This paradigm suggests that a dimensional model which is built on neurochemical and neuroanatomical circuitry will yield greater insights in treatment as they are based upon fundamental biological underpinnings.

Virtually all brain disorders may cause psychiatric symptoms. The term “neuropsychiatric disorders” may refer to brain disease or dysfunction that causes psychiatric symptoms. Examples of neuropsychiatric disorders include depression (including treatment resistant depression, bipolar depression, etc . . . ), schizophrenia, PTSD and other anxiety disorders, autism, ADHD, and the like. Even neurodegenerative disorders, such as dementia may present with primary psychiatric symptoms, especially in the earlier stages of the disease. As another example, the significant co morbidity of PTSD and Closed head injuries, especially in veterans, may present with primary psychiatric symptomatology.

Although various research and clinical studies have looked for diagnostic and therapeutic indicators to refine diagnosis and treatment, in an almost overwhelming variety of genomic markers, gene expression markers and protein markers, this vast and growing body of data has proven difficult to interpret. Most physicians are unable to synthesize the tremendous amount of information on possible risk factors and indicators in order to apply this information clinically to diagnose and/or treat patients. Thus, there is an as yet unmet need for reports, panels and/or kits that would allow a medical professional to apply the most relevant genetic, epigenetic, transcriptomic, proteomic and functional imaging tests in a meaningful manner to their patients. It is also critical to provide a proper analysis that allows the medical profession to understand and interpret the results of such tests, as well as have a resource to call upon for clarification of their interpretations.

This analysis, which can be referred to as ‘the Interpretation” may be useful in developing and understanding new sites of action, association, and/or patient response to psychotropic drugs, medical foods, lifestyle recommendations or other interventions for these disorders which otherwise is entirely random and arbitrary. The vast number of raw data in the neurosciences; Genomic variation, such as single nucleotide polymorphisms (SNPs), small tandem repeats (STRs), variable tandem repeats (VNTRs), copy number variants (CNVs), insertion/deletions (indels), rare variants, chromosomal duplications/deletions, CpG islands and shores, allele specific methylation, and the like, throughout the genome, as well as in specific genes, gene families, and/or pathways visualized by brain imaging procedures, are related to subtypes of psychiatric disorders, and the relative response to different classes of therapeutic agents, suggests that there is a growing need to provide an interpretation of information provided by genetic testing (particularly multiple genetic tests) to the clinician, or learned intermediary, to aid in treatment and/or diagnosis. Unfortunately, without providing a proper context, genomic test results can lead to confusion rather than clarification, particularly in a clinical setting. The heterogeneous nature of gene findings in neuropsychiatric disorders suggests that neuropsychiatric disorders themselves are heterogeneous and require a dimensional, rather than categorical approach, in agreement with NIH suggested. By analyzing disorders using a spectrum of biomarkers, such as SNP-based gene analysis, subtypes of neuropsychiatric conditions can be differentiated and treated in a personalized manner. This analysis may allow a deeper understanding of a patient's health across a variety of neuropsychiatric categories and will allow mental health professionals to treat individuals with more specific and targeted interventions. Therefore, the approach I am proposing to interpret biomarker data, such as gene expression can help identify subpopulations of patients that can benefit from more targeted pharmacotherapy, medical foods or other non-pharmacological interventions. Personalized medicine is considered a young but rapidly advancing field of healthcare that is informed by each person's unique clinical, genomic, and environmental information. Because these factors are different for every person, the nature of diseases—including their onset, their course, and how they might respond to drugs or other interventions—is as individual as the people who have them. The goal of personalized medicine is to customize or individualize treatments based on the particular environmental, genomic profile, and clinical information specific to a patient, thereby allowing accurate predictions to be made about a person's susceptibility of developing disease, the course of disease, and its response to treatment. In order for personalized medicine to be used effectively by healthcare providers and their patients, these findings must be translated into precise diagnostic tests and targeted therapies.

Treatment resistance in psychiatry is an area where there is a particular pressing need to provide biomarker-based tests that collects relevant biomarkers and presents the results of these biomarkers to a physician in an interpreted manner. As a specific example, within a 15-month period after having been diagnosed with depression, sufferers are four times more likely to die as those who do not have depression. Almost 60% of suicides have their roots in major depression, and 15% of those admitted to a psychiatric hospital for depression eventually kill themselves. In the U.S. alone, the estimated economic costs for depression in 1990 exceeded $44 billion. The World Health Organization estimates that major depression is the fourth most important cause worldwide of loss in disability-adjusted life years, and will be the second most important cause by 2020.

“Theranostic” information, using specifically identified biomarkers, such as genes or proteins associated with brain imbalances, may allow tailored treatment of neuropsychiatric disorders. The interpretive information provided may include a score or weighting index indicating a confidence level for the interpretive information. This could be anticipated to include a description of a common phenotype paired to a cluster of parallel biomarkers, and a scoring system which allows the clinician to equate the biological and non-biological variables in a way which produces a more integrative picture (phenotype-genotype based report).Both the testing and the report may be configured to extract patient information most relevant to treatment in order to facilitate the proper administration of said medical foods. It is anticipated that relevant neurobiological data may include an analysis of the actual protein expression of an altered gene through proteomics, DNA methylation analysis, including the methylation status of CpG islands, histones, or allele specific methylation, which may correlate with the activity of transcribed genes. Because CpG motifs are potentially modifiable by environmental factors, they provide a plausible biomarker by which a medical food or other medical intervention may be tracked to determine the effects of such intervention on relevant gene expression. This epigenetic analysis is an especially important methodology to assess both the changes in DNA expression as a consequence of a particular intervention longitudinally.

Methylation is also being explored as an indicator of drug response. For example, in breast cancer, low methylation of PITX2 in lymph nodes may predict recurrence after Tamoxifen treatment, and in glioblastoma, MGMT methylation may predict response to alkylating agents.

The “methylation density” can therefore represent an important element of this helping to elucidate biomarker relevance in neuropsychiatric disorders. DNA methylation patterns are surprisingly correlative across many somatic tissues, including brain and lymphocytes. For example, in an examination that included lymphocytes, inter-tissue correlation of 0.95, and suggesting substantial validity for peripheral measurement of DNA methylation as a surrogate for brain methylation status. Specific examples of methylation analysis as a “post intervention” biomarker could include an analysis of COMT promoter CpG islands, which have been detected to be hypomethylated in DNA derived from the saliva in schizophrenia. Further, the observation that S-adenosyl methionine is effective in ameliorating aggressive symptomatology in schizophrenic patients with low catechol-o-methyltransferase (COMT Met158Met variants), supports the notion that a specific medical food compound may exert its therapeutic effects through changes in the methylation of DNA. Methylation status, including hypo- and hyper-methylation of certain genes may be used as markers of neuropsychiatric disorder. Gene-specific methylation patterns may offer potential molecular signatures of medical food responsiveness in psychiatric disorders and may serve as a viable approach for revealing epigenetic processes.

### SUMMARY OF THE DISCLOSURE

This invention relates to methods of diagnosing and treating people with neuropsychiatric conditions non pharmacologically, using a previously undisclosed portfolio of medical food products including components that are combined in the medical food to enhance their effects in ways which otherwise would not be realized by a single compound. Additionally, the method of diagnosis discloses an alternative classification system which is also new, and which helps to better understand individuals with neuropsychiatric complaints without having to rely on the limitations of the current DSM. The methods described herein use genetic variants to probe the neurobiology of brain regions which mediate attention and executive brain function, limbic and hypothalamic function associated with arousal, anxiety and depression, hippocampal function associated with cognition, cognitive resilience, connections between cognitive-emotional centers and memory, and subcortical brain regions associated with emotional valence. This latter dimensional analysis is particularly relevant to assess addiction risk and other compulsions. These discrete brain regions as mentioned above will herein be referred to as Axis, I, II, III, and IV. Each of these, as will be disclosed, correlates with a particular neurotransmitter system (which may be altered in excess or deficiency), a distinct clinical phenotype, a cluster of genetic variants which parallel the metabolic causes of specific dysfunction as well as pairing of these axes with specific non pharmacological interventions; primarily, but not limited to medical foods.

Thus, a further and vital element of this disclosure relates not only how to optimize interpretation of said algorithm, rather it sets forth specific treatment algorithms which are primarily, but not exclusively, medically food based. Thus, an integral element of this discovery is the disclosure of combinations of specific dietary factors which may ameliorate the adverse metabolic consequences of such gene variants, in such a fashion as otherwise would not be achieved by a single agent.

For example, described herein are methods and compositions to treat neuropsychiatric disorders based upon a new framework of diagnosis. Axis I biomarkers include genes related to prefrontal dopamine synthesis and/or dopamine degradation. Axis II includes genes related to re uptake of dopamine, norepinephrine and serotonin and autonomic hyperactivity. Axis III includes genes relates to impairments in inflammatory pathways, glutamate neurotransmission and/or neurotrophic factors. Axis IV includes genes related to glutamate reuptake and predisposition to addictive behavior, and obsessive compulsions. Medical compositions for Axis I include agents designed to enhance prefrontal dopamine, including phophatidylserine and methylfolate. Axis II medical food interventions are agents designed to reduce excessive catecholamine activity by promoting reuptake mechanisms, reducing cortisol and/or promoting degradation of catecholamines and includes creatine, magnesium and taurine. Axis III medical foods are designed to reduce inflammation and promote neurotrophic factors and includes DHA conjoined to cytidine diphosphate choline. Axis IV medical foods include N acetylcysteine and sulphorophanes.

### DETAILED DESCRIPTION

Described herein are methods of treating a patient, including methods and apparatus for determining if a subject would benefit from one or more medical foods. In particular, describe herein are methods of performing a dimensional assay of a patient, including one or more biomarkers in each of four axes. In general a biomarker may include a genetic marker, including a polymorphism, an epigenic marker (e.g., methylation) or other post-intervention biomarker, a behavior biomarker (phenotype), or the like. This method may be automated using a processor that is configured to perform the analysis and provide output including a description of one or more medical food appropriate for the subject.

Examples of methylation analysis as a “post intervention” biomarker can be anticipated as markers of epigenetics; the influence of a specific intervention on the methylation of a gene or group of genes. These could include analysis of the methylation status of the serotonin transporter, COMT, MTHFR and other genes. The methylation level of the 5-HTT promoter region can cause effects of 5-HTTLPR on 5-HTT mRNA production. The methylation status, including hypo- and hyper-methylation of certain genes may be a biomarker informing the medical food component(s) that may be of use for treatment of a neuropsychiatric disorder. In general, epigenetic modulations may play an important role in fine-tuning of gene expression in response to environmental factors. Methylation may also be used as a biomarker for heightened stress, intractable anxiety and other clusters of symptoms related to the amygdala-HPA axis. For example, SLC6A4 methylation levels appear to modify the effect of the number of traumatic events on PTSD after controlling for SLC6A4 genotype. Persons with more traumatic events were at increased risk for PTSD, but only at lower methylation levels. At higher methylation levels, individuals with more traumatic events were protected from this disorder. Depressive symptoms are more common among those with elevated buccal cell 5HTT methylation who carried 5-HTTLPR short-allele. Thus hypomethylation of SLC6A4 may be used as a marker of depression and/or PTSD as well as being applied to track the response of a medical food intervention as a “surrogate marker” of response.

A step of presenting a weighted index of confidence level for the interpretive analysis of a neuropsychiatric domain analysis may be summarized in the report in a key, or they may be self-qualifying (e.g., the index may indicate “high,” “medium” or “low” confidence values).

As mentioned above, the interpretive analysis may further comprise a description of the physiological significance of the biomarker test result for the patient, a description of published studies describing similar biomarker test results, an indicator of which lifestyle interventions, medical foods and the like which may be most particularly important to prescribe to a given set of individuals, and/or a visual representation of a brain region affected by the biomarker. In any of the biomarkers described related to the 4 axis model herein proposed, additional polymorphisms affecting function of the gene (e.g., at the protein, methylation and/or nucleotide level) may be included and tested for; the same medical food component may be indicated. Thus, unless otherwise indicated, it may be the dysfunction of the gene, and not the specific polymorphism examined that indicates the medical food or medical food component.

The specific algorithm of using biomarker based data to create a “mind map” has been developed by the inventor. This map incorporates regional brain domains of significance useful for clinical purposes. While similar in principal to the “research domain criteria” suggested by the NIH, the specific genetic, proteomic or epigenetic data discussed specifically herein correlates with recommendations for individualized and non-pharmacological treatment strategies as actionable and practical steps to take as a result of presenting such data to the clinician or subject. Further, a new and previously undisclosed discovery relates to unique combinations of medical foods that can be prescribed to address specific neuropsychiatric dysfunction based upon the model disclosed herein.

The methods and articles of manufacture described herein may also be used to provide customized guidance for non-traditional therapeutics such as medical foods, herbal remedies, specific dietary strategies, exercise programs, styles of psychotherapy, and the like.

As used herein the phrase “medical food” may refer to foods that are formulated and intended for the dietary management of a disease or disorder. These foods may provide distinctive nutritional elements that cannot be met by normal diet alone. Medical foods may be distinct from the broader category of foods for special dietary use and from traditional foods that bear a health claim. A medical food may be a food for oral ingestion or tube feeding (nasogastric tube), may be labeled for the dietary management of a specific medical disorder, disease or condition for which there are distinctive nutritional requirements, and may be intended to be used under medical supervision. Examples of medical foods may include: nutritionally complete formulas, nutritionally incomplete formulas, and formulas for metabolic disorders. Although the variations and examples described herein are specific to medical foods, in some variations the compositions described herein may be prepared and/or compounded as traditional “drugs” or medicaments.

Alternative and non-conventional practices of medicine may include the use of such therapies (particularly in psychiatry), and it would be beneficial to include interpretive information on such non-traditional therapeutics. In some variations the neuropsychiatric panel may determine the state of one or more of these biomarkers, and may suggest a medical food component on the basis of the biomarker results. In some variations, the proposed individualized medical food may include multiple medical food components that may be combined into one (or more) medical foods; in some variations medical food components may be provided as separate medical foods.

Described below are exemplary biomarkers and medical food components corresponding to each biomarker as well as exemplary interpretive comments regarding the brain mapping data and the appropriate medical food to mitigate the specific abnormalities detected using such methods.

### Axis I: Attention Axis and Prefrontal Dopamine

The first axis (Axis I) may also be referred to as the attention axis or the prefrontal dopamine axis. A patient having a disruption in the prefrontal dopamine (axis I) axis may be identified using one or more biomarkers indicating an irregularity in dopamine transmission in the prefrontal cortical regions of the brain. Biomarkers may include genetic markers (SNPs), epigenic markers (methylation, etc.), and statistically significant identified (including self-identified) behaviors. Behaviors indicating a deficit in the first axis may include attention difficulty, poor focus, reduced ability to plan, impulsivity, motivational issues, and depression. When behavior is used as a marker, it may be combined with one or more other biomarkers (e.g., non-behavioral) biomarkers, such as genetic (SNP) indicators.

Axis I: Prefrontal brain regions
Genetic markers associated with Axis I including makers for dopamine
dysregulation; symptoms may include attention difficulty, poor focus,
reduced ability to plan, impulsivity, motivational issues, depression

| Related Genes | Exemplary SNP/ALLELE (biomarker) |
|---|---|
| 1) COMT | 472 G > A (Val158Met), rs4680 |
| 2) MTHFR variants | 677, and other methylation impaired |
| | pathways |

### Detailed Description of Axis I Diagnosis and Treatment

The first axis relates to neuropsychiatric conditions which are associated with the prefrontal cortex. The prefrontal cortex is referred to as the “executive brain” because it is involved in decision making, impulse control, the ability to plan for the future and judgment. The neurochemical basis of the prefrontal lobe is influenced, in large part, by the neurotransmitter dopamine. Dopamine levels are under both environmental and genetic control. A key enzyme that regulates prefrontal dopamine is catechol methyltransferase or COMT. As will be discussed, excess or reduced activity of COMT as a result of specific genetic factors results in a consistent set of behavioral dysfunction due to changes in prefrontal dopamine levels.

Activity of COMT is correlated with many critical biological functions, including cognition, stress response, and pain sensitivity. Changes in these biological functions in the brain are related, among other factors, to differences in circulating levels of catecholamines, primarily dopamine and norepinephrine. Depression, pain and stress may be caused by (or may co-exist along with) either relatively high or low levels of these catecholamines.

The prefrontal dopamine axis (Axis I) is generally concerned with regulation of cortical/frontal lobe dopamine systems. Biomarkers related to disruptions in this axis may include markers for one or more of: COMT (e.g. SNPs) and MTHFR. The catechol-O-methyltransferase (COMT) Val (158) allele in rs4680 (G allele) is associated with differential enzyme activity—specifically enhanced activity with parallel reductions in dopamine in the prefrontal lobes during working memory (WM). COMT is an enzyme involved in the degradation of dopamine, predominantly in the frontal cortex. Several polymorphisms in the COMT gene have been associated with poor cognition, diminished working memory, and increased ADD like symptoms as a consequence of altered dopamine catabolism. Suitable COMT gene polymorphisms include the functional common polymorphism (Val (158) Met; rs4680) but may include other nucleotide substitutions, changes in protein levels or epigenetic changes in methylation patterns.

The COMT rs4680 G/G genotype (Val/Val homozygous genotype) confers a significant risk of worse response after 4-6 weeks of antidepressant treatment in patients with major depression. There is a negative influence of the higher activity COMT rs4680rs4680 G/G genotype on serotonergic based antidepressant treatment response during the first 6 weeks of pharmacological treatment in major depression, possibly conferred by decreased dopamine availability. This suggests a potentially beneficial effect of interventions for such individuals who display this variant with medically food based dopamine agonists. Medical foods which are tailored to such individuals would include generally regarded as safe compounds (GRAS) which help to restore normal brain dopamine levels by reducing the activity of COMT in genetically prone individuals with high endogenous COMT and subsequently lower dopamine.

For example, a medical food formula for individuals having a disruption of the first axis such as that associated with COMT val genotype may include phosphatidylserine and methylfolate. In addition, the medical food may include Quercetin or EGCG, although these are not essential.

A mechanism whereby phosphatidylserine (PS), alone or in combination with methylfolate, may be used to improve ADD and other cognitive symptoms associated with low dopamine has not been previously disclosed. PS levels from subjects are inversely correlated with COMT enzyme activity, measured in lymphoblast cells. This finding indicates that excessive COMT activity, as found in individuals with val/val variants, adversely influences phosphatidylserine metabolism. Potentially, the higher the COMT activity an individual possesses, the greater the accompanying reduction in PS synthetic capacity. This suggests a functional relationship between COMT activity and the ability to synthesize PS. Thus, in people with high COMT enzyme function due to Val/val variants, lower levels of phosphatidylserine may be synthesized, requiring exogenous administration of the compound as a medical food to compensate for the adverse metabolic consequences resulting from the gene polymorphism. It has not been previously disclosed to prescribe phosphatidylserine, an endogenous phospholipid used herein as a medical food, based upon detection of such gene COMT val/val variants.

Clinically, PS has been used to address cognitive dysfunction in ADD and cognitive decline, but its mechanism of action to benefit these conditions has not been elucidated. For instance, the efficacy of PS was seen in a 15-week, double-blind, placebo-controlled trial, Two hundred ADHD children were randomized to receive either PS—or placebo. Efficacy was assessed using Conners' parent and teacher rating scales. The key finding of the double-blind phase was the significant reduction in the Global:Restless/impulsive subscale and significant improvement in Parent impact-emotional (PE) subscale in the phosphatidylserine treated group. We herein propose that exploratory subgroup analysis of children with a more pronounced response to PS may be predicted based upon COMT val/val variants. The mechanism of efficacy of PS is particularly greater in COMT val/val variants as without the clinical intervention, individuals with ADHD and COMT val/val variants would otherwise have lower PS brain levels.

Phosphatidylserine has also been shown to improve memory in elderly subjects, including some, but not all, double blind studies. The benefit of PS has been previously speculated to be due to improved brain lipid status, and never previously suggested that lower levels of PS as a result of COMt val variants being the primary reason for reduced cognition. Thus, cognitive disorders associated with low prefrontal dopamine resulting from COMT val/val mediated variants provides a new method to recognize and treat low phosphatidylserine levels by exogenous administration of the compound as a medical food in such identified individuals.

Thus, although PS may have been used in some patients, the methods described herein include the use of PS as a medical food specifically in those patients that may benefit from it the most, based on a disruption in the prefrontal dopamine axis as may be caused by COMT val/val polymorphism. Further, PS in these patients may be used in combination with methylfolate to provide synergistic results in this subpopulation of patients. Patients without an axis I deficiency, who may be less likely to benefit from PS and/or methylfolate may therefore be given other treatments, and/or not given a medical food including PS and/or methylfolate.

Methylfolate is another component of a medical food to address cognitive symptoms resulting from reduced brain dopamine as a result of excessive COMT activity and subsequently elevated levels of dopamine degradation. The combination of methylfolate and PS enhances the pharmacological effects of each in ways in which their benefit would not be actualized if administered separately. As proposed herein, individuals with a COMT Val/Val polymorphism may have especially impaired Axis I activity when found in conjunction with the MTHFR T/T variant:(Axis I—low dopamine) The axis I phenotype may be expressed secondary to reduced prefrontal dopamine as a consequence of these 2 genes being in epistasis, resulting in excess dopamine degradation due to lower methylation bioavailability, as normal methylation is required to suppress overactive COMT activity in val/val variants..

Common complaints seen in patients possessing both variants (COMT and MTHFR) include feelings of low motivation, depression, reduced energy, poor planning and disorganization. Thus, proposed medical food components may include phosphatidylserine and methylfolic acid based treatments.

Dysfunction of MTHFR (methylenetetrahydrofolate reductase) may result in reduced enzymatic activity of MTHFR, and thus impaired folic acid metabolism, and inefficient production of methylfolate. The 5,10-methylenetetrahydrofolate reductase (MTHFR) is a key enzyme for intracellular folate homeostasis and metabolism. Methylfolic acid, synthesized from folate by the enzyme MTHFR, is required for multiple biochemical effects in the brain. A primary role involves the synthesis of dopamine in the brain, due to its requirement in tyrosine hydroxylase—the rate limiting factor in dopamine synthesis. Folic acid deficiency results in fatigue, reduced energy and depression due to lower synthesis of dopamine. Low folate blood levels have been correlated with depression and polymorphisms of the MTHFR gene (e.g. rs1801133) are closely associated with risk of depression. The nucleotide 677 polymorphism in the MTHFR gene leading to a valine substitution at amino acid 222) encodes a thermolabile enzyme with reduced activity. The degree of enzyme thermolability (assessed as residual activity after heat inactivation) is much greater in T/T individuals (18-22%) compared with C/T (56%) and C/C (66-67%). MTHFR gene polymorphisms include polymorphisms in the 5,10-methylenetetrahydrofolate reductase (MTHFR) gene, including MTHFR C677T. These variants, which lead to reduced methylation capacity, are associated with common psychiatric symptoms including fatigue, poor concentration and depressed mood due to lower dopamine production.

The epistasis that is part of this disclosure involves further impairments of dopamine signaling as a result of hyperactive COMT activity found in val/val variants. Lower prefrontal dopamine is caused by three overlapping adverse metabolic effects as a consequence of these gene variants; 1) hypomethylation of COMT in MTHFR variants, which results in excessive breakdown dopamine through the COMT pathway, 2) Higher endogenous COMT, which enhances dopamine breakdown and 3) lower dopamine synthesis due to reduced methylation dependent tyrosine hydroxylase activity. In this model, COMT is disinhibited due to low methylation status, resulting in increased dopamine breakdown. Further, overactive COMT(val variants), the negative combined effects of low methylation as a consequence of the MTHFR variant leads to an even greater degree of dopamine degradation due to reduced methylation dependent suppression of COMT.

This can be summarized as a double hit hypothesis, uninhibited COMT and significant dopamine degradation as well as reduced dopamine synthesis due to the COMT val and MTHFR variants. It is hereby disclosed a new medical food composition designed to 1) inhibit COMT and reduce dopamine degradation 2) increase dopamine synthesis, and that this medical food be used primarily but not exclusively for COMT val/val and MTHFR variants who have Axis I symptomatology.

In the brain, PS requires methylation for synthesis. Phosphatidylserine in the brain is synthesized by base-exchange reaction and converted into lipid membranes through several enzymatic reactions leading to choline phosphoglycerides. The incorporation of phosphatidylserine into these downstream phospholipids requires successive methylations and normal bioavailability of methyl groups via enzymes such as phosphatidylethanolamine-N-methyltransferase. This observation strongly suggests that normal methylation enzyme activity is obligatory for phospholipid metabolism in the brain and conversely, impaired methylation may lead to neuropsychiatric symptoms as a result of disturbed phospholipid synthesis. Thus, the combination of phosphatidylserine with methylfolate may act synergistically to preserve and promote brain lipid membranes and dopamine signaling in the prefrontal cortex.

A potential additional way to correct excess COMT activity is through natural COMT inhibitors. Studies have shown that Epigallocatechin gallate (EGCG) and quercetin inhibit human COMT-mediated O-methylation of catechols via different mechanisms. Whereas EGCG inhibits COMT mainly through direct binding to the enzyme, quercetin (a catechol-containing bioflavonoid) inhibits COMT through direct competitive inhibition of the enzyme by serving as a substrate. A previously undisclosed use of phosphatidylserine in combination with these agents to inhibit excessive COMT while simultaneously raise phospholipids degraded by overactivity of the COMT enzyme. Quercetin and/or EGCG as synergistic agents with phosphatidylserine.

In some variations, diagnosis of Axis I diagnosis and treatment includes identifying COMT val/val polymorphism or otherwise identifying lower prefrontal dopamine, especially when found in combination with MTHFR variants. Giving a patient having an axis I disruption a medical food as described herein may ameliorate the axis I disruption by restoring prefrontal dopamine levels and/or returning prefrontal dopamine levels to a more normal baseline.

In any of the medical foods for treating axis 1 described herein, the medical food may include a first compound that reduces degradation of dopamine and a second compound that increases synthesis of dopamine. Generally, the first compound may be Phosphatidylserine (PI). The dosage of PI dosage may vary from between about 100 mg-1000 mg. The second compound may be methylfolate. The dosage of methylfolate may be between about 800 mcg-15 mg/daily. In any of these variations, Quecetin and/or EGCG may also be used as part of the formulation of the medical food.

### Axis II: Limbic Hyerarousal

The second axis (Axis II) may also be referred to as the limbic hyperarousal axis or the autonomic hyperarousal axis.

Axis II: Genetic markers associated with Axis II include makers for
catecholamine excess; neuropsychiatric symptoms associated with
autonomic hyperarousal may include panic attacks, insomnia,
hypervigilance, fear, increased startle. Systemic symptoms include
pain, fibromyalgia, somatization and the like

| Gene | Exemplary SNP/ALLELE |
|---|---|
| SERT (SLC6A4) | Ins/del, rs25531 |
| FKBP5 | rs3800373, rs1360780 |
| COMT met/met | |

The second relevant “domain” is the limbic brain, including the amygdala and hypothalamus, which when overactive, result in anxiety, hypervigilance and panic. The autonomic hyperarousal axis (or autonomic arousal axis), herein referred to as Axis II. Axis II dysfunction is due to excessive catecholamine activity (in contrast to Axis I), particularly dopamine, but also norepinephrine and serotonin. Gene variants which lead to metabolic defects, including serotonin transporter defects and COMT met/met variants result in higher catecholamine activity. The higher level of catecholamines in synapses leads to increased limbic activity with parallel changes in anxiety and hypervigilance. Studies have reported amygdala hyperactivity associated with the 5-HTTLPR short allele, linking the genetic and neuroimaging lines of research and suggesting a mechanism whereby the short allele confers heightened stress risk.

A highly relevant concern for persons who experience stress related disorders is that they are frequently prescribed SSRIs, which are less likely to be of benefit in these people. Thus, an element of this disclosure relates to specific treatment recommendations for such individuals.

COMT rs4680 met/met: It is currently disclosed that the met/met variant results in a unique subset of psychiatric symptoms due to reduced catecholamine degradation, reduced GABA binding in the brain and higher circulating dopamine in critical brain regions. Catechol O-methyltransferase (COMT) is a metalloenzyme that metabolizes biologically active catechol-containing structures by methylation of a single hydroxyl group. A number of neurotransmitters contain a catecholamine moiety and are deactivated by COMT. The enzyme COMT inactivates the catecholamine neurotransmitters (dopamine, norepinephrine, epinephrine and serotonin). It catalyzes the transfer of a methyl group from its co-substrate S-adenosylmethionine (SAMe). SAMe and magnesium are both COMT co-substrates which facilitate the binding of catechol molecules to COMT, which facilitates the degradation of these neurotransmitters. This may explain why met variants of the COMT enzyme experience higher levels of stress. With reduced inactivation of these sympathetic catecholamines, symptoms of anxiety are likely to be more pronounced. By recognizing the relationship of COMT met/met variants and anxiety, new medical foods which act to enhance the degradation of dopamine, norepinephrine or even serotonin are disclosed.

COMT forms two binding pockets that accommodate the adenosine and methionine side-chains of SAMe. Prior to catechol binding, magnesium must first displace the positively charged amine group. Thus, both SAMe and magnesium are obligatory co-factors for COMT activity by maintaining COMT in an open conformation which can bind catecholamines. A common variation in its coding sequence leads to a substitution of methionine (Met) in the peptide sequence (commonly referred to as Val158Met). The Met variant leads to reduced COMT stability, with a 40% reduction in COMT activity. The reduced COMT activity is hypothesized to result in higher levels of synaptic catecholamine(s). Thus, the Val158Met substitution may impact the thermostability of the COMT protein and may reduce COMT enzymatic activity by more than one-half in human brain. The Val158Met variant leads to reduced COMT stability, with a concomitant lower level of COMT activity and higher level of circulating catecholamine(s), such as dopamine and norepinephrine. The clinical consequence of reduced COMT activity associated with the Met variant may include increases in certain states of depression, anxiety, panic attacks and pain. Subjects with the COMT met variant have also been found to possess association to pain sensitivity in various studies and exhibit stronger pain-related fMRI signals in a number of brain structures. In human studies, low COMT activity has been associated with increased sensitivity to clinical preoperative or postoperative pain, and may increase the risk for fibromyalgia. As it is well known that various pain states are associated with autonomic hyperactivity, stress and anxiety, and we hypothesize that genetic variants which lead to higher levels of catecholamines would be associated with increased pain states, as well as psychiatric symptoms.

Fibromyalgia is a chronic, generalized pain syndrome that affects the musculoskeletal system. It is characterized by widespread pain, the presence of multiple tender points, and fatigue and sleep disturbances without any structural or inflammatory cause. Psychiatric clusters of symptoms, including heightened vulnerability to the detrimental effects of stress have also been observed in people with the COMT met variant. One treatment possibility for patients with the reduced COMT activity due to the Met variant is to utilize the enzymatic co-substrates S-adenosyl methionine (SAMe) and/or magnesium to theoretically boost COMT function. The presence of the co-substrate SAMe stabilizes the COMT enzyme and thereby may improve its biological activity to reduce excessive catecholamines.

To that end a number of double-blind placebo-controlled trials have found SAMe to be superior to placebo and equal in efficacy to tricyclic antidepressants for treating major depressive disorder. One randomized clinical trial with fibromyalgia showed that pain, fatigue, quality of sleep and clinical fatigue and mood improved more among the SAMe-treated patients than the placebo group.

The presence of S-adenosylmethionine with an attachment to magnesium is that the combined molecule stabilizes the COMT enzyme and thereby may improve its biological activity to reduce excessive catecholamines. S-adenosylmethionine and magnesium are COMT co-substrates which stabilize the secondary structure of the108M s-COMT by a docking mechanism which facilitates the binding of catechols. The V108M (met variant) polymorphisms have been shown to decrease COMT stability and activity by specifically distorting the SAM-binding site. As mentioned, COMT forms two pockets that accommodate the adenosine and methionine side-chains of SAM. Within the 108M closed state of COMT, SAM cannot bind inside the active site and therefore methylation cannot occur. Prior to catechol binding, magnesium must first displace the positively charged amine group. Thus, both S-adenosylmethionine and magnesium are obligatory co-factors for COMT activity by maintaining COMT in an open conformation which can bind catechols such as dopamine and norepinephrine. Thus, the use of SAMe with magnesium may act synergistically to enhance COMT function in COMT met variants and thereby reduce excessive catecholamines. The use of COMT variants clinically may be particularly helpful to determine with a higher degree of predictability the benefit or possibility of using SAMe (e.g., a composition including S-adenosylmethionine and magnesium) successfully to treat patients with depression and/or fibromyalgia.

An additional biomarker germane to the “mind map” of axis II is a gene known as FKBP5. FKBP5 regulates the cortisol-binding affinity and nuclear translocation of the glucocorticoid receptor. FKBP5 is a glucocorticoid receptor-regulating co-chaperone of hsp-90 and plays a role in the regulation of the hypothalamic-pituitary-adrenal system and the pathophysiology of stress.

FK506 regulates glucocorticoid receptor (GR) sensitivity. When it is bound to the FKBP5 receptor complex, cortisol binds with lower affinity and nuclear translocation of the receptor is less efficient. FKBP5 expression is induced by glucocorticoid receptor activation, which provides an ultra-short feedback loop for GR-sensitivity. Polymorphisms the gene encoding FKBP5 have been shown to associate with differential GR activation, differences in GR sensitivity and stress hormone system regulation. Alleles associated with enhanced expression of FKBP5 following GR activation, lead to an increased GR resistance and decreased efficiency of the negative feedback of the stress hormone axis. This results in a prolongation of stress hormone system activation following exposure to stress. This disregulated stress response might be a risk factor for stress-related psychiatric disorders.

Thus, it is hereby disclosed that serotonin transporter defects resulting in higher synaptic serotonin, COMT met/met variants which result in higher dopamine and norepinephrine due to reduced COMT mediated degradation and FKBP5 variants which result in reduced cortisol feedback regulation, are associated with Axis II dysfunction. Phenotypically, Axis II leads to states of increased anxiety, lower pain threshold, feelings of helplessness and depression, and other core psychiatric symptoms associated with increased autonomic hyperactivity and hypothalamic-pituitary adrenal dysfunction. Medical foods which are uniquely designed to address these metabolic effects, including means to lower cortisol, enhance catecholamine degradation are disclosed. A new integrative approach to accomplish this is hereby disclosed.

It is currently increasingly being appreciated that mitochondrial dysfunction is associated with psychiatric and neurological disorders. While various theories have been proposed to explain the relationship of mitochondrial dysfunction to psychiatric phenomenology, it is currently disclosed that the primary mechanism involves reduced catecholamine reuptake as a result of mitochondrial dependent catecholamine transporters. Catecholamine transporters, including serotonin, norepinephrine and dopamine are ATP dependent, as energy is required to transport the neurotransmitter back into the presynaptic neuron. With reduced ATP secondary to lower mitochondrial function, reduced transporter activity occurs with reduced inactivation of neurotransmission. These metabolic defects are especially enhanced in genetic variations in which the variant already leads to higher synaptic catecholamines such as SERT S/S (serotonin) and COMT met/met (dopamine).

It is thereby necessary to disclose medical foods for psychiatry which enhance mitochondrial ATP to enhance ATP dependent reuptake through transporter function. Mitochondrial enhancers for such individuals may include creatine, choline, magnesium and others.

As mentioned above, elevated cortisol is frequently encountered in psychiatric patients but there is currently no established method to lower cortisol as an efficacious agent in psychiatry. One such agent is a medical food called choline. higher oral choline intake, on average 1 gram daily, favorably impacts the epigenetic state of cortisol-regulating genes, and their expression, resulting in promoter methylation of the cortisol-regulating genes, corticotropin releasing hormone, glucocorticoid receptor; lower CRH transcript abundance (P=0.04); lower blood leukocyte promoter methylation of CRH and lower plasma cortisol., suggesting that choline intake in humans modulates the epigenetic state of genes that regulate HPA axis reactivity. Thus, in genetically prone individuals with higher circulating cortisol, the administration of choline may reduce cortisol production by inhibiting CRH. Choline may be especially important to consider in people with FKBP5 variants, in which cortisol levels are higher due to impaired degradation of cortisol. Choline may be part of the formulation in doses between 500 mg-2 gms/daily.

Creatine may also be contemplated as part of this disclosure, being used in said composition for individuals with depression and co morbid pain symptoms, muscle pain and the like. Since creatine reduces methylation demand as well as increasing mitochondrial ATP. The donor of methyl groups for almost all cellular methylation reactions is S-adenosylmethionine. SAMe catalyzes the synthesis of creatine and phosphatidylcholine. This may help explain the observed anti-depressant effects of SAMe, which increases brain creatine synthesis. As creatine is a downstream metabolic byproduct of SAMe, in genetically prone individuals with COMT met variants, administration of creatine may be an ideal substitute for SAMe. Like SAMe, creatine improves fibromyalgia and depressive symptoms. In depression, there are many studies in the literature which demonstrate creatine efficacy. For instance, 31-Phosphorus magnetic resonance spectroscopy ((31) P MRS) is a translational method for in vivo measurement of brain energy metabolites. Patients with mitochondrial disorders can present with primary psychiatric symptomatology, including mood disorder, cognitive impairment, psychosis, and anxiety. The most common psychiatric presentations in the cases of mitochondrial disorders included mood disorders, which have shown to be corrected by creatine augmentation of mitochondrial activity.

Compared to healthy controls, creatine-treated adolescents with SSRI failure demonstrated a significant increase in brain Phosphocreatine (PCr) concentration (31)P MRS brain scans.

There are also several controlled trials involving the efficacy of creatine in fibromyalgia. After 8 weeks of receiving creatine, significant improvement in parameters reflecting severity of fibromyalgia, quality of life and sleep, disability, and pain.

An additional and previously undisclosed use of creatine is for use in patients with primary deficits in catecholamine transporters, such as serotonin transporter short allele patients. Short transporter alleles have been associated with higher cortisol levels, reduced synaptic serotonin reuptake and higher rates of anxiety disorders. The defect in serotonin reuptake may be due to a mitochondrial impairment in transporter function. Catecholamine reuptake requires mitochondrial ATP. Creatine enhances mitochondrial ATP production and may act as an antidepressant in serotonin transporter short alleles, as well.

In addition, magnesium is an essential component of this disclosure to improve mitochondrial dysfunction in psychiatric patients, in particular Axis II phenotypes and genotypes in which excessive anxiety and pain syndromes are caused by overactive catecholamines.

Dietary Mg²⁺ deficiency is more prevalent than recognized and can cause psychiatric symptoms. Elevations in plasma catecholamines associated with an Axis II variants state are accompanied by hypomagnesemia, Magnesium is an obligatory mineral which enhances ATP production in the brain. The regulation of high-affinity uptake of NE, DA and serotonin by divalent cations, such as magnesium and ATP, is not commonly appreciated clinically. Inhibition of catecholamine uptake is observed Mg2+ is depleted from cell cultures. While this has been observed in vitro, the clinical application of magnesium to enhance catecholamine reuptake in anxiety states, has not been previously disclosed.

An ideal and preferred embodiment of magnesium administration includes the Mg²⁺ attached to the amino acid taurine. Taurine can suppress the release of adrenomedullary catecholamines and also acts as a cofactor in mitochondrial function.

As mentioned above, S adenosylmethionine may be another medical food to address Axis II by promoting COMT activity and thereby enhancing dopamine degradation. SAMe antidepressant effect has also been correlated with improved bioenergetics in depressed states. On MRS studies, SAMe increases creatine and brain ATP.

Thus, the following is disclosed. Axis II is a state of heightened autonomic activity due to high circulating catecholamines, high HPA axis activity, high cortisol, high anxiety and pain states. Gene variants which reduce catecholamine uptake(SERT s/s), reduce cortisol inactivation(FKBP5) and/or reduce catecholamine degradation(COMT met/met) are helpful to clinically identify and understand these variants to treat them appropriately. Additional markers of mitochondrial dysfunction may also be helpful as reduced ATP will lead to reduced transporter protein function.

Medical food compounds are disclosed which are comprised of creatine, magnesium (especially magnesium taurinate), choline and SAMe can be used. As doses of these agents are generally high to achieve a therapeutic effect,

The preferred embodiment is creatine, magnesium, taurine in equimolar concentrations. These doses may vary from 333 mg of each(creatine 333 mg, magnesium 333 mg, taurine 333 mg) up to a maximum dose of creatine 5 grams, magnesium 1 gram and taurine 1gm daily.

### Axis III: Cortical Glutamate Axis

The cortical and emotion brain axis (Axis III) may also be referred to as the glutamategic axis or the cortical glutamate axis. This axis is also pro-inflammatory. Dysfunction of in Axis III may be determined by one more biomarkers, including, for example, biomarkers for CACNA1C and BDNF.

Axis III: Genetic markers associated with disturbances in excitatory
neurotransmission due to glutamate dis-regulation, symptoms may
include heightened irritability, cyclical and recurrent mood disturbances,
paroxysmal complaints, reduced ‘neuroresilience’ and a
‘disconnect’ between cognitive and emotional brain
centers characterized by ‘irrational” behaviors

| Gene | Exemplary SNP/ALLELE |
|---|---|
| CACNA1C | G > A, rs1006737 |
| BDNF | G > A (Val166Met), rs6265 |

A third domain of significance relates to the hippocampus and its effects on cognition. There is a significant clinical need to have an assessment of cognitive resilience. Cognitive dysfunction, as a result of stress is a well-known phenomenon. However, there is a dearth of data related to how best to mitigate against these deleterious effects.

Key abnormal synaptic pathways of Axis III may indicate proneness to paroxysmal disturbances, irritability, instability, neurodegenerative vulnerability, migraine, pain and the like. This axis may be probed with biomarkers to the glutamatergic pathway (e.g., NMDA and AMPA receptors) as well as calcium ion channels. For example, CACNA1C is thought to be important in modifying the effects of: synaptic activity on cell survival, synaptic plasticity, MAPK pathway activation and critical pathways involved in learning and memory. Intracellular calcium levels are regulated specifically by CACNA1C which play a role in learning and memory via mediating the downstream effects of glutamate neurotransmission. CACNA1C mRNA levels increase following repeated amphetamine administration, and CACNA1C may be elevated in postmortem brains from BP patients. Preclinical and clinical studies support a role for CACNA1C in mood disorder pathophysiology, treatment resistance, autism and schizophrenia. Biomarkers implicated in regulation of glutamate may include calcium channel SNPs (e.g., rs2370419, rs1006736). The presence of these biomarkers may suggest that treatment with a non-pharmacological calcium channel antagonist, a specific medical food as outlined below, may be therapeutic in such patients.. Other biomarkers related to hippocampal function/dysfunction include gene variants of BDNF (Val66Met), BDNF protein levels or methylation of BDNF.

The calcium ion is one of the most versatile, ancient, and universal of biological signaling molecules, known to regulate physiological systems at every level from membrane potential and ion transporters to kinases and transcription factors. Disruptions of intracellular calcium homeostasis underlie a host of emerging diseases, the calciumopathies. Cytosolic calcium signals originate either as extracellular calcium enters through plasma membrane ion channels or from the release of an intracellular store in the endoplasmic reticulum (ER) via inositol triphosphate receptor and ryanodine receptor channels. Therefore, to a large extent, calciumopathies represent a subset of the channelopathies, but include regulatory pathways and the mitochondria, the major intracellular calcium repository that dynamically participates with the ER stores in calcium signaling, thereby integrating cellular energy metabolism into these pathways, a process of emerging importance in the analysis of the neurodegenerative and neuropsychiatric diseases. The CACNA1C gene encodes one subunit of a calcium channel. Results suggest that ion channelopathies may be involved in the pathogenesis of bipolar disorder, schizophrenia and autism with an overlap in their pathogenesis based upon disturbances in brain calcium channels. CACNA1C encodes for the voltage-dependent calcium channel L-type, alpha 1c subunit. Gene variants in CACNA1 (e.g. rs1006737) are associated with altered calcium gating and excessive neuronal depolarization. CACNA1 polymorphisms have been associated with increased risk of bipolar disease and schizophrenia. Psychiatric disease phenotypes, such as schizophrenia, bipolar disease, recurrent depression and autism, produce a constitutionally hyperexcitable neuronal state that is susceptible to periodic decompensations. The gene families and genetic lesions underlying these disorders may converge on CACNA1C, which encodes the voltage gated calcium channel. These findings suggest some degree of overlap in the biological underpinnings of susceptibility to mental illness across the clinical spectrum of mood and psychotic disorders, and show that at least some loci can have a relatively general effect on susceptibility to diagnostic categories based upon alterations in calcium signaling.

Clinically, there is a significant need to recommend safe, non-pharmacological alternatives to patients who display this variant. A specific medical food composition is disclosed herein which address this. In particular, described herein are methods of treating a deficiency in axis III in patients having such a deficiency comprising prescribing a cholinergic agonist such as DHA, cytidine diphosphate choline, and in some variations other medium chain fatty acids (e.g., decenoic acid).

As mentioned, biomarkers for Axis III deficiencies include BDNF biomarkers such as SNP/variant rs6265 Val/Met (G/A).

Brain-derived neurotrophic factor (BDNF) is a member of the nerve growth factor family. It is induced by cortical neurons and is necessary for neurogenesis and neuronal plasticity. BDNF has been shown to potentially mediate the effects of antidepressant treatment on neurogenesis and neuronal survival within the hippocampus.

The met variant of the BDNF gene has been associated with higher circulating cortisol and reduced hippocampal volume. A single nucleotide polymorphism in the BDNF gene has been identified causing a valine (val) to methionine (met) substitution at codon 66 in the prodomain (BDNFmet). Cultured rat hippocampal neurons transfected with the met allele have impaired BDNF production and distribution in the hippocampus, reduced dendritic arborization and lower c-Fos expression compared to rats with the val/val allele. BDNF may be relevant to the pathophysiology of major depression, and specifically related to stress mediated impairments in neurogenesis. The BDNF Val66Met variant is associated with hippocampal atrophy in abnormal mood states. Prolonged exposure to chronic stress may decrease BDNF production and reduce hippocampal volumes in genetically prone individuals. Older individuals with the BDNF met allele may be at an increased risk for depressive syndromes and reduced hippocampal volumes as a consequence of chronic stress or depression. Exposure to stress causes dysfunctions in circuits connecting hippocampus and prefrontal cortex. BDNF is down-regulated after stress. Thus agents which promote BDNF are new mechanisms to treat stress induced alterations in the limbic system. Brain-derived neurotrophic factor is a member of the nerve growth factor family. It is induced by cortical neurons and is necessary neurogenesis and neuronal plasticity. BDNF has been shown to mediate the effects of repeated stress exposure and long term antidepressant treatment on neurogenesis and neuronal survival within the hippocampus. The BDNF Val66Met variant is associated with hippocampal dysfunction, anxiety, and depressive traits.

Previous genetic work has identified a potential association between a Val66Met polymorphism in the BDNF gene and bipolar disorder. Meta-analysis based on all original published association studies between the Val66Met polymorphism and bipolar disorder shows modest but statistically significant evidence for the association between the Val66Met polymorphism and bipolar disorder from 14 studies consisting of 4248 cases, 7080 control subjects and 858 nuclear families. The BDNF gene may play a role in the regulation of stress response and in the biology of depression and the expression of brain-derived neurotrophic factor (BDNF) may be a downstream target of various antidepressants.

Exposure to stress causes dysfunctions in circuits connecting hippocampus and prefrontal cortex. BDNF is down-regulated after stress. Psychological stress down-regulates a putative BDNF site. Thus agents which promote BDNF are new mechanisms to treat stress induced alterations in the limbic system. BDNF binds to and activates tyrosine kinases receptor (TrkB) to exert its effects.

BDNF variants are associated with some clinical phenotypes (such as reduced brain volume) and reduced BDNF function. The met variant of the BDNF gene has been associated with higher circulating cortisol. Hippocampal volume is influenced by neurotrophic factors, including BDNF, which is widely distributed in the adult brain. A single nucleotide polymorphism in the BDNF gene has been identified causing a valine (val) to methionine (met) substitution at codon 66 in the prodomain (BDNFmet). Cultured rat hippocampal neurons transfected with the met allele have impaired BDNF production and distribution in the hippocampus, reduced dendritic arborization and lower c-Fos expression compared to rats with the val/val allele. BDNF may be relevant to the pathophysiology of major depression, and specifically related to stress mediated impairments in neurogenesis. The BDNF Val66Met variant is associated with hippocampal atrophy in abnormal mood states. Prolonged exposure to chronic stress may decrease BDNF production and reduce hippocampal volumes in genetically prone individuals. Older individuals with the BDNF met allele may be at an increased risk for depressive syndromes and reduced hippocampal volumes as a consequence of chronic stress or depression.

Inflammation is commonly associated with psychiatric disorders, but the primary mechanism of inflammation and specific metabolic consequences of inflammation on brain pathways is still not fully understood. This disclosure discusses the inter relationship of inflammation, excessive calcium ion activity, NMDA receptor hyperactivity and consequent reduced levels of BDNF as being linked to PLA2. PLA2, a phospholipase, degrades membrane lipids in the brain. Therapies aimed at reducing PLA2 activity, especially in Axis III variants are hereby disclosed.

Phospholipase A2 significantly decreases glutamate uptake, primarily through an arachidonic acid mediated pathway. As glutamate is the primary neurotransmitter involved in bipolar pathogenesis, one mechanism which links inflammation to bipolar states involves excessive PLA2. In fact, PLA2 levels have been observed to be higher in bipolar patients in several clinical studies.

Subset of bipolar I disorder patients with a history of psychosis have elevated calcium-independent PLA2 activity. Given that this enzyme activity is also increased in schizophrenia, elevated rates of phospholipid turnover mediated by the enzyme could represent a common biochemical feature of psychotic illness.

Thus, Axis III can be characterized as a state of “inflammation”, both systemic and neurological. This inflammation may be expressed clinically by symptoms of persistent agitation, irritability, headache pain and aggression. Gene variants, including the CACNA1C gene which results in excessive glutamate mediated depolarization is associated with the phenomenology of bipolar. Excessive glutamate activity, as a consequence of inflammatory mediated increased PLA2 and calcium mediated NMDA depolarization, leads to hippocampal brain damage.

Migraine and bipolar disorders are episodic disorders that share many clinical features and underlying pathophysiological mechanisms. As mentioned, disruptions of intracellular calcium and sodium homeostasis may underlie a host of neuropsychiatric disorders including bipolar, epilepsy and migraine; genetically based disruptions of intracellular calcium and/or sodium homeostasis may result in a constitutionally hyperexcitable state that is susceptible to periodic decompensations, a clinical aspect which characterizes all three conditions.

More often than noticed, bipolar disorder occurs together with migraine. In a study of patients suffering from bipolar disorder (both type I and II), comorbid migraine was found in 24.5% of all bipolar cases and similar studies have found comorbidity in 24.8% (versus a general population rate of 10.3%). Insights into commonalities in the pathophysiology of these disorders may suggest new treatment approaches for both conditions. Strong support for a shared genetic basis comes from familial hemiplegic migraine (FHM), an autosomal dominant syndrome characterized by severe migraine, that arises as a result of mutations in genes for the membrane ion transport proteins CACNA1A (P/Q-type voltage-gated calcium channel). Cortical spreading depression (CSD), a wave of profound cellular depolarization in migraine is often preceded by glutatamate mediated cellular hyperexcitability associated with localized epileptiform discharges. Glutamate is a critical mediator of the hyperexcitability in migraine. Many antiepileptic drugs prevent the occurrence of migraine attacks and are also used to treat bipolar disorder, supporting the view that neuronal hyperexcitability is a common pathophysiological mechanism in both disorders.

Results suggest that polymorphisms in the gene and subsequent disturbances in brain calcium channels are involved in the pathogenesis of bipolar disorder, migraines, schizophrenia and autism. The convergence of these conditions may be etiologically linked by a heritable, constitutionally hyperexcitable neuronal state due to calcium mediated neuronal depolarization in limbic brain regions. The gene families and genetic lesions underlying these disorders may converge on CACNA1C, which encodes the voltage gated calcium channel, suggesting some degree of overlap in the biological underpinnings of susceptibility to mental illness across the clinical spectrum of mood and psychotic disorders. Further, glutamate transmission is strongly dependent on calcium homeostasis and on mitochondrial function. Glutamate excitotoxicity has been associated with elevated expression of VGCCs and enhanced voltage-gated calcium currents, mitochondrial dysfunction, implicating mitochondrial dysfunction in VGCC-mediated cell death. Neurological dysfunction in Migraine and bipolar are linked by calcium overload, sodium channel dysregulation, excess NMDA receptor activity and mitochondrial dysfunction.

Individuals most vulnerable to hippocampal damage as a result of BDNF polymorphisms.

A medical food composition is hereby disclosed that inhibits PLA2, reduces NMDA neurotoxicity and enhances BDNF function. The components of this disclosure include EPA and/or DHA(preferably DHA as will be disclosed below), Cytidine diphosphate choline and other medium chain fatty acids, such as decenoic acid.

### Medical Food Interventions (Axis III):

Dietary n-3 polyunsaturated fatty acids (e.g., ω-3 or n-3 PUFA, fish oils) are known to exhibit antiarrhythmic efficacy in vitro and in vivo. Animals administered n-3 PUFA exhibit reduction in heart rate and ischemia-induced ventricular fibrillation. Eicosapentaenoic (EPA) and Docosahexaenoic (DHA) acids have been shown to decrease electrical excitability and the antiarrhythmic effect of n-3 PUFA is related to inhibition of Na+ and/or Ca2+ currents. For example, DHA decreases peak L-type Ca2+ current in ventricular myocytes and causes voltage-dependent block of L-type Ca2+ current.

Recent evidence indicates that long-chain polyunsaturated fatty acids (PUFAs) can prevent cardiac arrhythmias by a reduction of cardiomyocyte excitability. This was shown to be due to a modulation of the voltage-dependent inactivation of both sodium (Na) and calcium (ICa) currents. The EC50 for the shift of the INa steady-state inactivation curve was 2.1+/−0.4 microM for docosahexaenoic acid (DHA) and 4+/−0.4 microM for eicosapentaenoic acid (EPA). The EC50 for the shift on the ICa inactivation curve was 2.1+/−0.4 for DHA and >15 microM for EPA. Additionally, DHA and EPA suppressed both INa and ICa amplitude at concentrations >10 microM. Thus, DHA has 2× potency to reduce excessive calcium mediated depolarization.

DHA also acts to inhibit PLA2, which may explain its efficacy in bipolar.

A link between omega-3 fatty acids and mood disorders is suggested by some studies showing a lower incidence of depression among populations with a diet rich in omega-3 fatty acids. Lower plasma levels of omega-3 fatty acids have been reported in mood disorder patients compared to healthy controls. Recent meta-analysis confirmed that depression is associated with lower levels of total PUFAs and both types of omega-3 PUFAs, eicosapentaenoic acid (EPA) and docosahexaenoic acid (DHA) may exert anti depressive effects. Clinical depression can be accompanied by low levels of omega-3 PUFAs in RBC, plasma and, as more found recently, brain tissue. In a recent double blind study, Omega-3 augmentation of citalopram treatment produced a significantly greater reduction in HAM-D scores as compared to citalopram treatment alone. In bipolar disorder, a meta-analysis was completed which was comprised of 5 pooled datasets (n=291) on the outcome of bipolar depression. A significant effect in favor of omega-3 (P=0.029), with a moderate effect size of 0.34 provided strong evidence that bipolar depressive symptoms may be improved by adjunctive use of omega-3 fatty acids. The anti-depression efficacy of Omega 3 fatty acids may be secondary to both stabilization of calcium mediated NMDA receptor activity and reduced PLA2 activity.

In addition to omega 3 fatty acids and magnesium, other fatty acids comprising a medical food would be of expected benefit to treat neuropsychiatric states associated with CACNA1C and/or BDNF variants. This includes 2 decenoic acid, an unsaturated fatty acid unique to royal jelly (RJ), protects against depression and anxiety in various animal models

2 Decenoic acid is a medium-chain fatty acids (MCFAs) with 8-12 carbons. These esters facilitate activation (phosphorylation) of extracellular signal-regulated kinases 1 and 2 (ERK1/2) and causes the activation of intracellular signal molecules other than ERK1/2 such as Akt and cAMP-responsive element binding protein (CREB), a transcription factor, all of which promote BDNF. The ability of 2 decenoic acid to enhance BDNF makes the compound an ideal agent to address BDNF variants.

Thereby, the Medical food Component and/or Intervention for CACNA1Crs1006737 variant and/or BDNF variants should include Omega3 Fatty Acids conjugated in ways known to those skilled in the art with medium chain fatty acids such as 2 decenoic acid, which together act to reduce neuronal excitability through ion channel stabilization and increase BDNF.

Lower plasma levels of omega-3 fatty acids have been reported in mood disorder patients compared to healthy controls, and these lower blood levels may disinhibit calcium channel activity in genetically prone individuals.. Recent meta-analysis confirmed that depression is associated with lower levels of total PUFAs and both types of omega-3 PUFAs, eicosapentaenoic acid (EPA) and docosahexaenoic acid (DHA) may exert anti depressive effects. Clinical depression can be accompanied by low levels of omega-3 PUFAs in RBC, plasma and, as more found recently, brain tissue. In a recent double blind study, Omega-3 augmentation of citalopram treatment produced a significantly greater reduction in HAM-D scores as compared to citalopram treatment alone. In bipolar disorder, a meta-analysis was completed which was comprised of 5 pooled datasets (n=291) on the outcome of bipolar depression. A significant effect in favor of omega-3 (P=0.029), with a moderate effect size of 0.34 provided strong evidence that bipolar depressive symptoms may be improved by adjunctive use of omega-3 fatty acids. What is disclosed in this invention is that the antidepressant effects of omega 3 fatty acids may be secondary to stabilization of calcium channels.

In addition to omega 3 fatty acids, and particularly DHa, CDP choline is an element of this disclosure, which acts in unexpected synergism with DHA. CDP choline, also referred to as citicholine reduces PLA2 activity. Hippocampal choline-containing compounds (Cho) determined with 1H MR spectroscopy (MRS) are decreased in major depression episodes and return to baseline levels after antidepressive treatment. Choline production inhibits the activity of various phospholipases significant inverse correlation (p=0.018) between PLA2 protein levels and MRS Cho/NAA levels suggesting a possible down regulation of PLA2 in response to choline.

The preferred composition to inhibit PLA2, inhibit calcium channels and increase BDNF is Sn1DHA-CDP choline. The SN1 form of DHA attached to choline more favorably crosses the blood brain barrier. The mg of DAH may vary from 100 mg-to 1 gram in SN-1 attachment to cytidine diphosphate choline, in doses between 500 mg-2 gms/daily. The composition can be used selectively or preferentially in Axis III variants, especially those with biomarkers such as elevated PLA2, BDNF met/met and CACNA1C. 2 deconic acid, a medium chain fatty acid, may also be regarded, but is not essential to the composition.

### Axis IV: Subcortical Glutamate Axis

The subcortical glutamate axis (Axis IV) may also be referred to as the emotional valance axis, the axis which mediates pleasure and pain. As described herein, dysfunctions in this axis may be indicated by a biomarker such as a polymorphism in one or more of SLC1A1, OPRM1 and DBH, which result in excessive glutamate.

| | | Medical food Component |
|---|---|---|
| Biomarker | SNP/variant | and/or Intervention |
| SLC1A1 | rs301430 G > T | N-acetyl L-cysteine (NAC) |
| | relate to glutamate transporter | |
| | dysfunction and thus | |
| | excessive synaptic glutamate; | |
| OPRM1 | | |
| DBH | | |

A disruption of axis IV may be indicated by the possession of genetic markers associated with emotional valence, subcortical (“basal ganglia/subcortical circuitry”) disruption, and particularly those influencing the balance of dopamine and glutamate neurotransmission. Phenotypes associated with this axis may include addiction predilection, anxiety, OCD, ruminations. This may be a very important domain to identify clinically because this region of the brain relates to emotional valence, both positive and negative. Positive valence relates to seeking out pleasurable experiences. The “feel good” effects of this brain region are related primarily to dopamine in the nucleus accumbens. Conversely, negative valence, also mediated by the same subcortical circuits, may be primarily mediated by the neurotransmitter glutamate. Thus, the balance between pleasure promoting dopamine and pain promoting glutamate in the subcortex is a critical element in addiction, anxiety disorders such as OCD, harm avoidance, and cravings.

The primary biomarkers related to these pathways are SLC1A1, a glutamate transporter that is inhibited by oxidative stress, and an opiate receptor gene, called OPRM1, and dopamine beta hydroxylase. The neurochemical deficits associated with these polymorphisms may unfavorably modulate dopamine receptor function under conditions of impaired glutathione synthesis as in substance abuse, addictions, schizophrenia and autism.

The DBH gene regulates plasma dopamine beta-hydroxylase activity (pDbetaH). A single nucleotide polymorphisms (SNPs), -1021C-->T (rs1611115) independently influence pDbetaH.

Dopamine-beta-hydroxylase (DbetaH) catalyzes the conversion of dopamine to norepinephrine in central noradrenergic and adrenergic neurons and thus is critically involved in the biosynthesis of catecholamines. DBH activity levels are altered in affective disorders, particularly in subtypes of affective disorders. The DBH TT genotype exhibited higher neuroticism underlying association of the TT genotype at DBH-1021 with impulsive personality traits. By balancing the ratios of dopamine and norepinephrine, dopamine beta hydroxylase (DBH) plays an important role in brain reward circuit that is involved with behavioral effects of addiction. DBH -1021C/T (rs1611115) is a functional variant with strong correlation with plasma DBH activity1021TT and carriers exhibit higher addictive predilection in some studies. For instance, seventy-four cocaine- and opioid-codependent (DSM-V) subjects were stabilized on methadone for 2 weeks and compared to placebo groups (n=40) for 10 weeks, genotyped for the DBH gene polymorphism, -1021C/T (rs1611115), that reduces DβH enzyme levels, and evaluated by examining the subject's capacity for increasing cocaine free urines. In addition to DBH, the OPRM1 gene has also been associated with addictive traits and variable response to Naltrexone for abstinence. Compared to controls, ADHD patients (with and without SUDs) showed significantly increased frequency of the DBH and the OPRM1 risk genotypes. The DBH risk genotype was associated with ADHD diagnosis, with the association strongest in the pure ADHD group. The OPRM1 risk genotype increased the risk for the combined ADHD and substance abuse phenotype.

Therapeutically, results show that ascorbate promptly enhances norepinephrine synthesis from dopamine by neuronal cells by promoting the activity of DBH. As DBH is a cofactor in the conversion of dopamine to norepinephrine, genetic variants may have an imbalance of dopamine and norepinephrine. Thus, ascorbic acid, by promoting DBH activity, may represent a previously undisclosed method to address individuals with this variant who experience certain psychiatric complaints.

Ascorbic acid is highly concentrated in the brain, and acts as a neuromodulator. The normal human brain has a vitamin C concentration of approximately 1 mM, 10 times the normal serum concentration. Once in the CSF, vitamin C appears to enter the brain interstitium by oxidation of ascorbate to DHA and uptake of DHA. Dehydroascorbate, the oxidized form of vitamin C, has been shown to stimulate the antioxidant defenses of cells, preferentially importing dehydroascorbate over ascorbate. DHA is the major transport form of ascorbate in cells, but within the cell DHA is regenerated into ascorbate at the expense of reduced glutathione (GSH). Unless reduced back to ascorbate, DHA is subsequently rapidly hydrolyzed into a 5-carbon sugar that can enter the nonoxidative enzymes pathways, thus DHA is not merely a transport form of vitamin C.

Ascorbate functions as a neuromodulator of both dopamine- and glutamate-mediated neurotransmission. Ascorbate deficiency is associated with significant increases (−25%) in dopamine levels. Following ascorbate treatment, striatal dopamine was further decreased, blocked an amphetamine-induced increase in stereotypy and the behavioral effects of amphetamine were also attenuated either by intraventricular or striatal infusions of ascorbate. The mechanism by which ascorbate acts to reduce dopamine may be via stimulation of dopamine beta hydroxylase. DBH is the rate limiting enzyme in the synthesis of norepinephrine from dopamine. By stimulating DBH, ascorbate reduces dopamine activity. Further, reduced levels of ascorbate may alter dopamine receptors, specifically changing the affinity of dopamine to the D2 receptor due to a redox mechanism.

A metabolic relationship between cysteine, glutathione and ascorbate/DHA redox couples has not been disclosed for clinical purposes. Ascorbate is stabilized by thiols, and decreased tissue ascorbate levels are found in GSH deficiency. Without co-administration, NAC may absorb all available ascorbate to re-establish its anti-oxidant status, leading reduced amounts of ascorbate to block excessive inflammatory pathways.

Deficiency of GSH leads to increased reduction of dehydroascorbate. Reduction of dehydroascorbic acid by GSH/NADPH dependent dehydroascorbic acid reductase decreases significantly in inflamed patients, suggesting that the capacity of the inflamed tissue to maintain the concentration of reduced ascorbic acid is also diminished. Deascorbate is the oxidized form of vitamin C. Administration of DHA leads to a 3× increase in cellular glutathione. DHA likely does this by stimulating the pentose phosphate pathway, leading to increased transaldolase, increased NADH and most importantly, maintains GSH levels by preserving sulfhydryl groups

As mentioned earlier, a key molecular imbalance associated with Axis IV dysfunction is oxidative stress, particularly in the basal ganglia. Oxidative stress in schizophrenia, OCD, addiction and autism has been detected in the subcortical brain regions in several animal models. These lines of evidence point to the utility of raising antioxidant brain defense systems to mitigate the risk of developing a childhood psychotic disorder such as schizophrenia or autism. In particular, glutathione activity may be neuroprotective in these disorders by its influence on receptor interactions within receptor heterodimers and receptor mosaics, representing an important integrative mechanism for signaling based upon redox sensitive mechanisms in brain networks.

The tripeptide, glutathione (gamma-glutamylcysteinylglycine) is the primary endogenous free radical scavenger in the brain. When glutathione (GSH) levels are reduced there is increased cellular oxidative stress, characterized by an increase and accruement of reactive oxygen species (ROS). This may result in alterations in dopaminergic and glutamatergic activity implicated in these illnesses. Glutamate and dopamine are highly redox reactive molecules and produce free radicals during neurotransmission. Neurons are thus at high risk for oxidative injury and pro-oxidative states have detrimental consequences on normal migrational processes and brain connectivity during development. GSH is synthesized in two steps, catalyzed by two different enzymes. During the first step, gamma-glutamylcysteine synthetase (GCS) catalyzes the formation of L-gamma-glutamyl-L-cysteine from glutamate and cysteine. The second step incorporates glycine under influence of glutathione synthetase, yielding GSH. GSH content is dependent on the supply of NAC, sarcosine and glycine. A major part of glycine is utilized for the synthesis of glutathione in astroglial cells

Synthesis of glutathione, a major redox regulator, is compromised in schizophrenia. The glutathione deficit, via its effect on redox-sensitive proteins could contribute to dysfunction of neurotransmitter systems in schizophrenia. Experimental models of glutathione deficit changed the modulation of responses by dopamine, from enhanced responses in control neurons (likely via D1-type receptors) to decreased responses in low-glutathione neurons (via D2-type receptors). The effect of a glutathione deficit on dopamine signaling was dependent on the redox-sensitive ryanodine receptors (RyRs), whose function was enhanced in low-glutathione neurons. This suggests that enhanced RyRs in low-glutathione neurons strengthens intracellular calcium-dependent pathways following activation of D2-type receptors and causes a decrease in function of L-type channels. This represents a mechanism by which dopaminergic systems could be dysfunctional under conditions of impaired glutathione synthesis as in schizophrenia. These changes closely mimic the pathological imbalances of dopamine signaling in schizophrenia, where D1 receptor function is blunted and D2 receptor activity is exaggerated.

N-acetyl cysteine (NAC) is a precursor of cysteine and glutathione. It has antioxidant properties, lipid stabilization, and preservation of mitochondrial membrane potential, all of which may favorably impact receptor function in neuropsychiatric states. Treatment of neurons with lipid peroxidation byproducts results in a drastic reduction of mitochondrial membrane potential, and this reduction is prevented by NAC. This neuroprotective effect is due, at least in part, to preservation of mitochondrial membrane potential and intracellular GSH levels. Thus, NAC may exert neuroprotective effects via its ability to inhibit oxidation of mitochondrial proteins, and stabilization of receptor membrane dimers.

NAC is also a potent glutamate modulator in the brain via its effects on the glutamate/cystine antiporter. The glutamate/cystine antiporter x(c)- transports cystine into cells in exchange for glutamate at a ratio of 1:1. Glutamate exported by system x(c)- is largely responsible for the extracellular glutamate concentration in the brain, whereas the imported cystine is required for the synthesis of the major endogenous antioxidant, glutathione. System x(c)- thus connects the antioxidant defense with neurotransmission and behavior. Disturbances in the function of system x(c)- have been implicated in nerve cell death due to increased extracellular glutamate and reduced intracellular glutathione. In vitro, inhibition of cystine import through system x(c)- leads to cell death by a mechanism called oxidative glutamate toxicity, which includes depletion of intracellular glutathione, activation of 12-lipoxygenase, accumulation of intracellular peroxides, and the activation of a cyclic guanosine monophosphate (cGMP)-dependent calcium channel towards the end of the death cascade. N-acetyl cysteine (NAC) inhibits glutamate via the cystine-glutamate exchange system. Further, by boosting glutathione, NAC acts as a potent antioxidant and has been shown in two positive, large-scale randomized placebo-controlled trials to affect negative symptoms in schizophrenia and depression in bipolar disorder.

N-acetylcysteine (NAC) treatment exerts its effects by activating cystine-glutamate exchange. NAC is also a potent glutamate modulator in the brain via its effects on the glutamate/cystine antiporter. The glutamate/cystine antiporter x(c)- transports cystine into cells in exchange for glutamate at a ratio of 1:1. Glutamate exported by system x(c)- is largely responsible for the extracellular glutamate concentration in the brain, whereas the imported cystine is required for the synthesis of the major endogenous antioxidant, glutathione. System x(c)- thus connects the antioxidant defense with neurotransmission and behavior. Disturbances in the function of system x(c)- have been implicated in nerve cell death due to increased extracellular glutamate and reduced intracellular glutathione. In vitro, inhibition of cystine import through system x(c)- leads to cell death by a mechanism called oxidative glutamate toxicity, which includes depletion of intracellular glutathione. N-acetyl cysteine (NAC) inhibits glutamate via the cystine-glutamate exchange system. Further, by boosting glutathione, NAC acts as a potent antioxidant and has been shown in two positive, large-scale randomized placebo-controlled trials to affect negative symptoms in schizophrenia and depression in bipolar disorder.

N-acetylcysteine (NAC) treatment exerts its effects by activating cystine-glutamate exchange and thereby stimulating extrasynaptic metabotropic glutamate receptors (mGluR).

Thus, as part of this disclosure, the clinical decision to use NAC can be based by detection of polymorphisms the SLC1A1 gene which regulates glial glutamate reuptake. The solute carrier family 1 member 1 (SLC1A1) regulates the glutamate transporter. Disturbances in the function of system x(c)- have been implicated in nerve cell death due to increased extracellular glutamate and reduced intracellular glutathione. The T allele of the SLC1A1 has been associated with reduced activity of the transporter, resulting in excess glutamate neurotransmission. Higher synaptic glutamate is associated with some psychiatric subendophenotypes, including OCD and autism (autism spectrum disorder, ASD). A medical food component, NAC, has demonstrated efficacy in some OCD and autistic patients. The inventor herein proposes that the subset of patient's with positive outcomes on NAC may be those having the SNP variant rs301430 G>T, as NAC may modulate the SLC1A1 glutamate transporter.

In genetically prone individuals with reduced glutamate/cysteine antiporter activity, the subsequent metabolic defect may be ameliorated through the administration of the amino acid N acetylcysteine (NAC). N-acetylcysteine may reduce excess glutamate activity by enhancing cysteine uptake in neurons via the glutamate/cysteine antiporter. N-acetyl cysteine (NAC) inhibits glutamate via the cystine-glutamate exchange system. N-acetyl cysteine (NAC) is a precursor of cysteine and glutathione. While N-acetylcysteine has demonstrated efficacy in OCD, autism and several other neuropsychiatric states via its effects by activating cystine-glutamate exchange and down regulating excess glutamate neurotransmission, instructing its use by detecting variants in the SLC1A1 gene have not been previously disclosed.

Sarcosine is also contemplated as a potential medical food component for gene variants associated with Axis IV. NMDARs are regulated in vivo by the amino acids glycine and D-serine. Sarcosine, a potent glycine transporter inhibitor, can increase synaptic glycine and promote NMDAR function. Potentiation of the N-methyl-D: -aspartate (NMDA) receptor glycine site, activation of group II mGluR, and activation of glutamate-cysteine antiporters, are the therapeutic aspect of this invention. Medical food or pharmacological manipulation of these specific NMDA receptor subtypes are recognized as being potentially as being efficacious in the treatment of schizophrenia and autism. Sarcosine, also known as N-methylglycine, is an intermediate and byproduct in glycine synthesis and degradation. Sarcosine is an amino acid involved in one-carbon metabolism and a promising therapy for schizophrenia, autism and other psychotic disorders characterized by impaired NMDA receptor function because it enhances NMDA receptor (NMDAR) function by inhibiting glycine uptake. Sarcosine is an NMDAR co-agonist at the glycine binding site. Sarcosine is metabolized to glycine by the enzyme sarcosine dehydogenase, while glycine methyl transferase generates sarcosine from glycine. Sarcosine is a natural amino acid and plays a significant role in various physiological processes and is the prime metabolic source glutathione. Sarcosine is a potent glycine transporter inhibitor and can increase synaptic glycine and promote NMDAR function. Sarcosine and N-acetylcysteine both ameliorated PPI deficits in mGluR5 knockout mice, pointing to their utility as treatments in schizophrenia. The antipsychotic potential of sarcosine is supported by its ability to restore the prepulse inhibition (PPI) deficit, hyperlocomotion and regional brain c-Fos expression changes caused by an NMDAR antagonist, ketamine. It has been suggested that serine (e.g., D-serine) may be modulated. For example, U.S. patent application Ser. No. 13/210,808, filed on Aug. 16, 2011 (US-2012-0041066), and titled “MEDICAL FOODS FOR THE TREATMENT OF DEVELOPMENTALLY-BASED NEUROPSYCHIATRIC DISORDERS VIA MODULATION OF BRAIN GLYCINE AND GLUTATHIONE PATHWAYS,” which was previously incorporated by reference, describes the use of NAC and sarcosine to treat prodromal schizophrenia, likely by increasing synaptic glycine and promoting NMDAR function.

Sarcosine, also known as N-methylglycine, is an intermediate and byproduct in glycine synthesis and degradation. Glycine acts as a precursor for serine, which functions as a co-agonist of NMDA receptors. Sarcosine is an amino acid involved in one-carbon metabolism and a promising therapy for schizophrenia, autism and other psychotic disorders characterized by impaired NMDA receptor function because it enhances NMDA receptor (NMDAR) function by inhibiting glycine uptake. Sarcosine is an NMDAR co-agonist at the glycine binding site.

Sarcosine is metabolized to glycine by the enzyme sarcosine dehydrogenase, while glycine methyl transferase generates sarcosine from glycine. Sarcosine is a natural amino acid and plays a significant role in various physiological processes and is the prime metabolic source glutathione. Sarcosine is a potent glycine transporter inhibitor and can increase synaptic glycine and promote NMDAR function. Sarcosine and N-acetylcysteine both ameliorated prepulse inhibition (PPI) deficits in mGluR5 knockout mice, pointing to their utility as treatments in schizophrenia. The antipsychotic potential of sarcosine is supported by its ability to restore the prepulse inhibition deficit, hyperlocomotion and regional brain c-Fos expression changes caused by an NMDAR antagonist, ketamine.

The combination of Sarcosine and N-acetylcysteine has not been previously described, and can be used as a medical food to treat these disorders.

In addition to NAC, Sulphorophanes, may also be used as medical food components. For example, astrocytes support neuronal antioxidant capacity by releasing glutathione, which is cleaved to cysteine in brain. Free cysteine is then taken up by neurons through EAAT3 (SLC1A1) to support glutathione synthesis. Activation of the nuclear factor erythroid 2-related factor 2 (Nrf2)-antioxidant responsive element (ARE) pathway promotes astrocyte release of glutathione, and Nrf2 activators enhance EAAT3 transcriptional activation. Since Nrf2 is believed to be induced by sulphoraphane, sulphoraphane may therefore increase EAAT3. Thus, in addition to NAC as a regulator/augmenter of SLC1A1, sulphorophanes can also be used clinically to treat individuals with evidence of excess glutamate secondary to SLC1A1 polymorphisms.

The combination of NAC and sulphorphanes to treat addictive conditions or cravings has not been previously disclosed. NAC use for reducing addictions may be beneficial.

Treatment with N-acetylcysteine (NAC) normalizes glutamate (Glu) homeostasis and prevents relapse in drug-dependent animals. In an open-label, randomized, crossover study of 8 cocaine-dependent patients and 14 healthy controls, one group receiving no compound and the other following a single administration of 2400 mg NAC. After administration of NAC, Glu levels were reduced in the cocaine-dependent group, whereas NAC had no effect in healthy controls. In a double-blind, placebo-controlled trial, 15 participants received N-acetylcysteine or placebo. While taking N-acetylcysteine, participants reported less desire to use and less interest in response to cocaine.

Methamphetamine (METH) is a powerfully addictive stimulant associated with serious health conditions. Pretreatment with sulphorphanes at 1, 3, and 10 mg/kg elicited a dose-dependent attenuation of acute hyperlocomotion in mice, after a single administration of METH, as well as lowering of DA levels and DOPAC in the striatum.

Thus, one medical food composition includes both N-acetylcysteine (NAC) and sulforophanes. Ascorbic acid and sarcosine may or may not be included in the composition. Ascorbic acid can be applied clinically as a separate intervention in DBH individuals.

Thus, treatment for Axis IV (e.g., primarily SLC1A1 individuals having a biomarker for a polymorphism in SLC1A1), may include N-acetylcysteine between about 500 mg-2400 mg/daily and Sulforophane between about 5-70 mg/daily. In some variations, the medical food also includes one or both sarcosine and ascorbate.

Each of the medical food embodiments corresponding to their respective axis can be formulated in ways which improve their bioavailability, solubility and passage across the blood brain barrier. An often overlooked facet in non-pharmacological interventions to treat neurological or psychiatric considerations is the failure to consider the ability of said compounds to cross the blood brain barrier. Thus, exemplary embodiments of each of these discloses could include “tight junction modulators”. Tight junction modulators change the charge of the molecule to facilitate transfer across the BBB. Examples could include cyclodextrin, chitosans, esters and amide salts. Further, excipients which increase solubility can be contemplated in each embodiment. Delayed release or extended release formulations may be preferred, owing to the persistence of stable psychiatric symptoms. pH triggered enteric coatings, or natural agents which change the stomach or intestinal pH, all of which can favorably increase AUC may also be effectively used.

The diagnostic assessment tool herein disclosed may be paired to non-pharmacological interventions and may also include additional dietary, life style and other interventions directed at improving the quality of life of such impaired individuals. Thus, cognitive behavioral therapies or other suggestions can be regarded where appropriate, sleep hygiene measures, acupuncture, physical therapy modalities, NeuroEEG biofeedback and the like can be anticipated to be uniquely designed and crafted to address the 4 axis domain dysfunctions.

### EXAMPLES

In one example, a patient visited her doctor because of complaints of poor focusing. Additional complaints include an inability to complete tasks, depression characterized by low energy and motivation, poor planning and being disorganized. Testing revealed a COMT val/val variant and MTHFR variant (e.g., Axis I). Because of this result, a medical food is prescribed, comprised primarily of phosphatidylserine 300 mg daily and 5 mg of methylfolate used concurrently as a daily dose. As described above, methylfolate works synergistically by inhibiting COMT and reducing dopamine degradation. Self-reported symptom improvement included better attention, ability to complete tasks and brighter mood.

In a second example, a patient complained of frequent bouts of irritability and depression. Symptoms may be associated with sleep disturbances and occasionally migraine headaches. Anti-depressants previously made symptoms significantly worse. Gene analysis revealed variants in the CACNA1C gene and BDNF (e.g., Axis II). Because of the symptoms and gene testing results, a combination of ethyl EPA 1 gm daily, mixed with 2 decenoic acid and 400 mg magnesium taurinate were included in a medical food for the patient. These compounds worked synergistically to reduce overactive calcium ion activity and enhance BDNF. Migraine symptoms and depression improved, and reports of better memory were also noted.

In a third example, a patient complained of feeling frequently stressed and overwhelmed. He frequently feels worse during deadlines and other pressures. In addition to self-reported stress, a diagnosis of fibromyalgia was made previously. SSRI were mildly effective. A blood test revealed elevated nocturnal cortisol. Gene testing revealed a serotonin short transporter and a COMT met/met variant, indicating overactive HPA axis and limbic hyperarousal (e.g., axis II). The patient was prescribed a medical food composed of Creatine 500 mg 3×/day, magnesium and Phosphatidylcholine 1 gm daily. Within 2 weeks, self-reported stress levels were markedly reduced and symptoms of fibromyalgia were improved.

In a fourth example, a child with developmental delay was seen by his provider. Core symptoms include OCD behavior and some perseverations. Family history was remarkable for addictions and OCD. Gene testing revealed a variant in SLC1A1 (e.g., Axis IV). N acetylcysteine was prescribed (500 gms/daily) and ascorbic acid (1 gm daily). Obsessions improved, including hair pulling.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of treating a patient having a disruption in a prefrontal dopamine axis (Axis I) with a medical food, the method comprising providing the patient with a medical food composition comprising a first compound that reduces degradation of dopamine and a second compound that increases synthesis of dopamine.

2. The method of claim 1, further comprising determining that the patient has a disruption in the prefrontal dopamine axis.

3. The method of claim 1, further comprising determining that the patient has a disruption in the prefrontal dopamine axis based by identifying one or more biomarkers indicating a disruption in the prefrontal dopamine axis.

4. The method of claim 1, further comprising determining that the patient has a disruption in the prefrontal dopamine axis based by identifying a disruption in the patient's catechol methyltransferase pathway(s).

5. The method of claim 1, further comprising determining that the patient has a disruption in the prefrontal dopamine axis based by identifying a polymorphism in the patient's COMT gene, MTHFR gene or both COMT and MTHFR genes leading to a decrease in dopamine.

6. The method of claim 1, wherein providing the first compound that reduces degradation of dopamine comprises phosphatidylserine.

7. The method of claim 1, wherein providing the second compound that increases synthesis of dopamine comprises methylfolate.

8. The method of claim 1, wherein providing the patient with a medical food composition comprises providing the patient with a medical food composition comprising phosphatidylserine and methylfolate.

9. The method of claim 1, wherein providing the patient with a medical food composition comprises providing the patient with a medical food composition comprising phosphatidylserine and methylfolate, epigallocatechin gallate (EGCG) and quercetin.

10. A method of treating a patient having a disruption in a prefrontal dopamine axis (Axis I) with a medical food, the method comprising:
determining that the patient has a disruption in the prefrontal dopamine axis based on the presence of one or more biomarkers indicating a disruption in the prefrontal dopamine axis; and
providing the patient with a medical food composition comprising phosphatidylserine and methylfolate that reduces degradation of dopamine and increases synthesis of dopamine.

11. A method of treating a patient having a disruption in a limbic hyperactivity (Axis II) with a medical food, the method comprising providing the patient with a medical food composition comprising a first compound that increases brain mitochondrial activity and a second compound that increases the degradation and/or reuptake of catecholamines.

12. The method of claim 11, further comprising determining that the patient has a disruption in the limbic hyperactivity axis based on the presence of one or more biomarkers indicating a disruption in the production or clearance of catecholamines in the brain.

13. The method of claim 11, wherein providing comprises providing a medical food including S-adenosyl methionine and magnesium.

14. The method of claim 11, wherein providing comprises providing a medical food including S-adenosyl methionine, magnesium, creatine and taurine.

15. The method of claim 11, further comprising determining that the patient has a disruption in the limbic hyperactivity axis based on a polymorphism in one or more of: COMT, SERT (SLC6A4), or PKBP5.

16. A method of treating a patient having a disruption in a limbic hyperactivity axis (Axis II) with a medical food, the method comprising:
determining that the patient has a disruption in the limbic hyperactivity axis based on the presence of one or more biomarkers indicating a disruption in the prefrontal dopamine axis; and
providing the patient with a medical food composition comprising a creatine, magnesium and taurine configured to increase brain mitochondrial activity and increase the degradation and/or reuptake of catecholamines.

17. A method of treating a patient having a disruption in a cortical glutamate axis (Axis III) with a medical food, the method comprising providing the patient with a medical food composition comprising a combination of compounds that inhibits PLA2, reduces NMDA neurotoxicity and enhances BDNF function.

18. The method of claim 17, further comprising determining that the patient has a disruption in the disruption in a cortical glutamate axis based on the presence of one or more biomarkers indicating a disruption in the calcium-dependent NMDA transmission.

19. The method of claim 17, further comprising determining that the patient has a disruption in the disruption in a cortical glutamate axis based on the presence of a polymorphism in one or more of CACNA1C and BDNF indicating a disruption in the calcium-dependent NMDA transmission.

20. The method of claim 17, wherein the medical food composition comprises docosahexaenoic acid (DHA) and cytidine diphosphate choline and one or more medium chain fatty acids.

21. The method of claim 17, wherein the medical food composition comprises docosahexaenoic acid (DHA), cytidine diphosphate choline and decenoic acid.

22. A method of treating a patient having a disruption in a cortical glutamate axis (Axis III) with a medical food, the method comprising:
determining that the patient has a disruption in the cortical glutamate axis based on the presence of one or more biomarkers indicating a disruption in the calcium-dependent NMDA transmission; and
providing the patient with a medical food composition comprising a combination of compounds that inhibits PLA2, reduces NMDA neurotoxicity and enhances BDNF function, the combination comprising docosahexaenoic acid (DHA) and cytidine diphosphate choline.

23. A method of treating a patient having a disruption in a subcortical glutamate axis (Axis IV) with a medical food, the method comprising providing a biomarkers indicating an excess of glutamate or a deficiency of dopamine in a subcortical region with a medical food composition comprising sulphoraphane and N-acetylcysteine.

24. The method of claim 23, further comprising determining that the patient has a disruption in the subcortical glutamate axis based on the presence of one or more biomarkers indicating an excess of glutamate or a deficiency of dopamine in a subcortical region.

25. The method of claim 23, wherein the medical food further comprises Sarcosine.

26. The method of claim 23, wherein the medical food further comprises ascorbate.

27. The method of claim 23, further comprising determining that the patient has a disruption in the subcortical glutamate axis based on the presence of one or more biomarkers indicating an excess of glutamate or a deficiency of dopamine in a subcortical region based on the presence of a polymorphism in one or more of SLC1A1, OPRM1 and DBH genes.

28. The method of claim 23, further comprising determining that the patient has a disruption in the subcortical glutamate axis based on the presence of one or more biomarkers indicating an excess of glutamate or a deficiency of dopamine in a subcortical region based on the presence of one or more biomarkers comprises identifying that the patient has the rs301430 G>T polymorphism in a SLC1A1 gene.

29. The method of claim 23, wherein the medical food comprises a dose of between about 500 and about 2400 mg/daily of N-acetylcysteine and a dose of between about 5 and about 70 mg/daily of sulphoraphane.

30. A method of treating a patient having a disruption in a subcortical glutamate axis (Axis IV) with a medical food, the method comprising:
determining that the patient has a disruption in the subcortical glutamate axis based on the presence of one or more biomarkers indicating an excess of glutamate or a deficiency of dopamine in a subcortical region comprising a polymorphism in one or more of SLC1A1, OPRM1 and DBH genes; and
providing the patient with a medical food composition comprising sulphoraphane at a dose of between about 5 and about 70 mg/daily and N-acetylcysteine at a dose of between about 500 and about 2400 mg/daily.
